(19) United States
(12) Patent Application Publication
(10) Pub. No.: US 2013/0266622 A1
(43) Pub. Date: Oct. 10, 2013

(54) Personal Care Articles Comprising Substrates and Personal Care Compositions and Methods of Making the Same

(71) Applicant: THE PROCTER & GAMBLE COMPANY, Cincinnati, OH (US)

(72) Inventors: Shawn David MCCONAUGHY, Cincinnati, OH (US); Edward Dewey SMITH, III, Mason, OH (US); Scott William SYFERT, Fort Mitchell, KY (US)

(73) Assignee: The Procter & Gamble Company, Cincinnati, OH (US)

(21) Appl. No.: 13/856,717

(22) Filed: Apr. 4, 2013

Related U.S. Application Data
(60) Provisional application No. 61/620,213, filed on Apr. 4, 2012.

(51) Int. Cl. A61K 8/11; B65B 3/04; A61Q 19/00

(52) U.S. Cl. CPC A61K 8/11; A61Q 19/00; B65B 3/04
USPC 424/401; 510/438; 510/404; 53/467

(57) ABSTRACT

A personal care article is disclosed having a substrate surrounding a personal care composition. Methods for making the personal care article and forming the personal care composition are also disclosed.

### TECHNICAL FIELD

The present disclosure generally relates to personal care articles, cleaning compositions, methods of manufacturing personal care articles, and methods of making a personal care composition.

### BACKGROUND

Cleansing the skin and/or hair is an activity that has been done for many years. Over time, skin and hair cleansing have involved the use of compositions such as bar and liquid soaps, body washes, shampoos, conditioners, and the like. For these compositions, consumers desire good cleansing properties and lathering characteristics, mildness toward the skin, and the ability to provide benefit agents to the target surface.

To enhance a consumer's experience, such cleansing compositions can also be coupled with implements such as a washcloth, a sponge, or a puff. For example, many consumers dispense liquid soaps or body washes onto a puff and then cleanse by applying the puff to their skin and/or hair. Similarly, many consumers rub bar soaps with a washcloth and then cleanse by applying the washcloth to their skin and/or hair.

Some attempts have been made to combine an implement with a cleansing composition in a personal care article. However, these executions were not ideal. For example, one such article included a rigid bar soap coupled with an implement. The rigidity of this type of execution does not conform to the surface to which it is applied and making it difficult to thoroughly clean the target surface. Some other attempts at a more conformable product did not provide a desired reusability and tended to create additional waste. In particular, such cleansing articles tend to lack durability and/or include cleansing compositions that completely dissolve after very few uses.

Accordingly, it would be desirable to provide a compliant personal care article having a cleansing composition that can have desirable cleansing properties, including suitable lathering and rinsing characteristics, can conform to the skin and/or hair, can be reusable and easy to use. Further, it would be desirable to provide an efficient and cost-effective method of making such personal care articles.

### SUMMARY OF THE INVENTION

In accordance with one embodiment, a personal care article comprises a substrate. The personal care article further comprises about 3,500% or greater, by weight of the substrate, of a personal care composition surrounded by the substrate. The personal care composition exhibits a complex viscosity from about 10 Pascal-seconds (Pa·s) to about 100,000 Pa·s and has a compliance value of about 0.01 kg/mm to about 1.5 kg/mm.

In accordance with yet another embodiment, a method of making a personal care article comprises providing a substrate, wherein the substrate comprises a compartment having an open end. The method further comprises filling the compartment with about 3,500% or greater, by weight of the substrate, of a personal care composition via the open end. The method further comprises sealing the open end such that the personal care composition is surrounded by the substrate. The personal care composition exhibits a complex viscosity from about 10 Pascal-seconds (Pa·s) to about 100,000 Pa·s and has a compliance value of about 0.01 kg/mm to about 1.5 kg/mm.

### BRIEF DESCRIPTION OF THE DRAWINGS

FIG. 1 depicts a perspective view of a personal care article according to one embodiment;

FIG. 2 depicts a side view of a personal care article according to one embodiment;

FIG. 3A depicts a cross sectional view of the personal care article of FIG. 2, along line 3-3;

FIG. 3B depicts a cross sectional view of the personal care article of FIG. 2, along line 3-3, where additional substrates have been added;

FIG. 4 depicts a side view of a personal care article according to another embodiment;

FIG. 5A depicts a cross sectional view of the personal care article of FIG. 4, along line 5-5;

FIG. 5B depicts a cross sectional view of the personal care article of FIG. 4, along line 5-5, where the composition is in the form of pellets.

### DETAILED DESCRIPTION OF THE INVENTION

Any application to which this application claims priority, like U.S. Application Ser. No. 61/620,213 filed Apr. 4, 2012, is incorporated herein by reference.

### I. DEFINITIONS

As used herein, the following terms shall have the meaning specified thereafter:

“Cleansing composition” refers to compositions intended for topical application to a surface such as skin and/or hair to, for example, remove dirt, oil, and the like. The cleansing compositions disclosed herein can be rinse-off formulations, in which the product is applied topically to the skin or hair via, for example, an implement or substrate and then subsequently rinsed within seconds to minutes from the skin and/or hair with water.

“Compliant” as used herein refers to an article and/or composition that can at least partially conform to a surface to which it is applied by some degree of deformation.

“g/use” refers to grams per use, which is the unit used for rate of consumption. The method for measuring and/or calculating the rate of consumption is described in U.S. Patent Application Pub. No. 2012/0246851.

“Macroapertured” refers to a substrate containing well-defined apertures having an average diameter of about 300 microns or greater. “Microapertured” generally refers to a substrate containing well-defined microscopic apertures (i.e., those not readily visible to a naked eye having 20/20 vision).

“Natural” refers to materials that can be derived from plants, animals, insects or materials that can be byproducts of plants, animals, and insects.

“Nonwoven” refers to a substrate comprising fibers not woven into a fabric but rather formed into a sheet. The fibers can either be random (i.e., randomly aligned) or the fibers can be non-random. For example, the nonwoven can be carded (i.e., combed to be oriented in primarily one direction).

“Personal care” refers to a composition or article for topical application to skin and/or hair. Personal care compositions can be rinse-off formulations, in which the composition can be applied topically to the skin and/or hair and then subsequently rinsed within seconds to minutes of application. The composition could also be wiped off using a substrate. The personal care articles or compositions can also be used for cleansing of the skin, cleansing of the hair, shave preparation, post shave treatment, or a combination thereof.

“Reusable” refers to an article that can be used for a number of usage events, such as showers and/or baths, wherein the number of usage events can be about 5 or greater, about 7 or greater, about 10 or greater, about 15 or greater, about 20 or greater, about 25 or greater, or about 30 or greater.

“Substantially free of” refers to about 5% or less, about 3% or less, about 1% or less, or about 0.1% or less of a stated ingredient. “Free of” refers to no detectable amount of the stated ingredient or thing.

“Substrate” refers to a material which can limit the amount of water to which a personal care composition is exposed during a usage event versus exposure of a personal care composition itself absent a substrate. The substrate may be, for example, a film, formed film, batting, woven, nonwoven, or a combination thereof.

“Synthetic” refers to materials that can be obtained primarily from various man-made materials or from natural materials which have been altered. “Usage event” refers to one 5 minute cycle of the Consumption Test described in U.S. Patent Application Pub. No. 2012/0246851.

“Water insoluble substrate” refers to a substrate which does not dissolve in water during the life of the article.

### II. PERSONAL CARE ARTICLES

Personal care compositions can come in many forms. One of the more common forms is bar soap. Bar soap can be generally non-compliant and rigid. The rigidity of most bar soaps can make them difficult to grip, making it more difficult to use during cleansing. Rigid bar soaps can also have the disadvantage in that only the surface which directly contacts the skin can be used for cleansing, and surface area can be limited by the bar's non-compliant nature. Conventional rigid bar soap can have a compliance value of about 2.5 kg/mm or above.

Bar soaps are often used with an implement, like a wash cloth. At some point, a wrapped bar article was formed by combining the implement and the bar soap. Many of these previous attempts at a wrapped bar article maintained the non-compliant bar and thus still suffered from its short comings. Additionally, adding a wrap to conventional bar soap also had the unintended effect of reducing the consumption rate of the bar making cleansing difficult due to reduced access to the soap. The reduction in consumption also made the product last too long which meant the substrate could wear out before the bar was used up.

Insoluble components in bar compositions can contribute to bar rigidity. Utilizing compliant compositions and/or articles can forgo a need for rigidity and can often result in elimination of the insoluble components giving a faster dissolving composition. And while it is desirable to have a composition that provides sufficient cleaning properties during a cleansing event (i.e. dissolves well enough to release surfactant), there should be a balance between such cleaning properties and longevity of the product. Otherwise, the product can be entirely consumed in just one or a few events and the product can be too expensive and wasteful to the environment.

How quickly a composition or article is used up by a consumer can be measured by its consumption rate, the method of which is described in U.S. Patent Application Pub. No. 2012/0246851. Substrates can be used to control the rate of consumption. So, when compositions dissolve quickly, substrates can be used to increase the consumption rate to a desirable level, like that of a typical bar soap. In certain embodiments, a substrate can act as a water flux limiting substrate to effectively increase the consumption rate. In certain embodiments, the water flux of the substrate can be manipulated by the chemical or physical make-up of the substrate. For example, the substrate can contain pores to allow more water passage. If less water passage is desired, the pore number can be reduced during manufacturing of the substrate or some of the pores can be sealed off.

Additionally, it is desirable to minimize the environmental impact of consumer products. Body wash can exceed 80% water, increasing transportation impact and requiring a significant amount of plastic packaging material. Most consumers use an implement of some kind to facilitate cleansing when using a body wash. For example, a pouf comprising a bunched polyolefin scrim weighing about 30 grams can be commonly used with body wash. The median lifetime of a pouf (in the U.S.) can be about 3 months; thus, the environmental impact of the pouf can be about 0.33 grams polyolefin/day of waste material generated. The total environmental impact of body wash and pouf can be considerably greater if packaging materials are included. Compliant articles can provide an improved environmental footprint compared to a pouf. Some compliant articles can provide half or even less plastic waste material per shower than a pouf.

A personal care article comprises a substrate and a personal care composition. The personal care article may also comprise multiple substrates. The personal care article may be used, for example, on skin, hair, or both. The personal care article may also be used, for example, for cleansing of the skin, cleansing of the hair, shave preparation, post shave treatment, or a combination thereof. In one embodiment, the personal care article is a personal cleansing article. In one embodiment, the personal care article is reusable.

The personal care article can be compliant (i.e. it at least partially conforms to a surface to which it is applied by some degree of deformation.) For example, if the article is a personal care article for cleansing the skin, then the article will bend to some degree to more fully contact a curved body part like the arm. Thus, if the personal care article is originally flat with no curve, when applied to the arm for cleansing there would be some amount of bend to better conform to the arm. Likewise, if the article's shape has a small amount of a curve, when applied to the arm the article would bend to some degree to more fully contact the arm. Oppositely, if the original article is curved such that it would not need to bend to conform to a curved surface like the arm, then it would bend to straighten when applied to a less curved surface like the abdomen. In one embodiment, the article and/or composition is fully compliant meaning it is capable of completely conforming to the surface to which it is applied.

In some embodiments, the compliant article will comprise a particulate composition. A particulate composition can be made of smaller particles like sand, larger particles like pellets, or anything in-between, and combinations thereof. These compositions may be formless and thus rely on a substrate or substrates to house them for use. For these types of articles, it is the ability of the composition in combination with the substrate(s) to at least partially deform to the shape of the surface to which it is applied that makes them compliant.

In some embodiments, compliance of the article can be measured according to the test described in more detail below. In some embodiments, a personal care article can comprise a compliance value of about 1.50 kg/mm or less. In varying embodiments, the compliance value of the article is about 1.35 kg/mm or less; about 1.25 or less; about 1.2 or less; about 1.1 or less; or about 1.0 or less. In additional embodiments, the article has a compliance of about 0.01 kg/mm to about 1.50 kg/mm; about 0.03 kg/mm to about 1.50 kg/mm; about 0.05 kg/mm to about 1.25 kg/mm; about 0.05 kg/mm to about 1.15kg/mm; about 0.10 to about 1.1; or any combination thereof.

The personal care composition can also be compliant similar to what is discussed above for the article. For example, if the composition is a personal cleansing composition for cleansing the skin, then the composition will bend to some degree to more fully contact a curved body part like the arm. Thus, if the personal cleansing composition is originally flat with no curve, when applied to the arm for cleansing there would be some amount of bend to better conform to the arm. Likewise, if the composition's shape has a small amount of a curve, when applied to the arm the composition would bend to some degree to more fully contact the arm. Oppositely, if the original composition is curved such that it would not need to bend to conform to a curved surface like the arm, then it would bend to straighten when applied to a less curved surface like the abdomen.

In some embodiments, compliance of the composition can be measured according to the test described in more detail below. In some embodiments, a personal care composition can comprise a compliance value of about 1.50 kg/mm or less. In varying embodiments, the compliance value of the composition is about 1.35 kg/mm or less; about 1.25 or less; about 1.2 or less; about 1.1 or less; or about 1.0 or less. In additional embodiments, the composition has a compliance of about 0.01 kg/mm to about 1.50 kg/mm; about 0.03 kg/mm to about 1.50 kg/mm; about 0.05 kg/mm to about 1.25 kg/mm; about 0.05 kg/mm to about 1.15kg/mm; about 0.10 to about 1.1; or any combination thereof.

In some embodiments, the composition and/or article may become compliant after exposure to water. Thus, you may have a non-compliant composition or article that, after exposure to a liquid, like water, during a usage event, becomes compliant. If an article and/or composition become compliant by the end of a second usage event, then they are considered compliant according to this application.

The personal care article will have a rate of consumption. This is a measure of how much of the composition is used during a usage event. A method for measuring consumption rate of the article is described in more detail below. In one embodiment, the article will have a consumption rate of about 20 g/use or less. In another embodiment, the article will have a consumption rate of about 15 g/use or less. In alternate embodiments, the article will have a consumption rate of about 1.5 g/use to about 15 g/use; from about 2.5 g/use to about 10 g/use; from about 3.5 g/use to about 6.5 g/use, or any combination thereof.

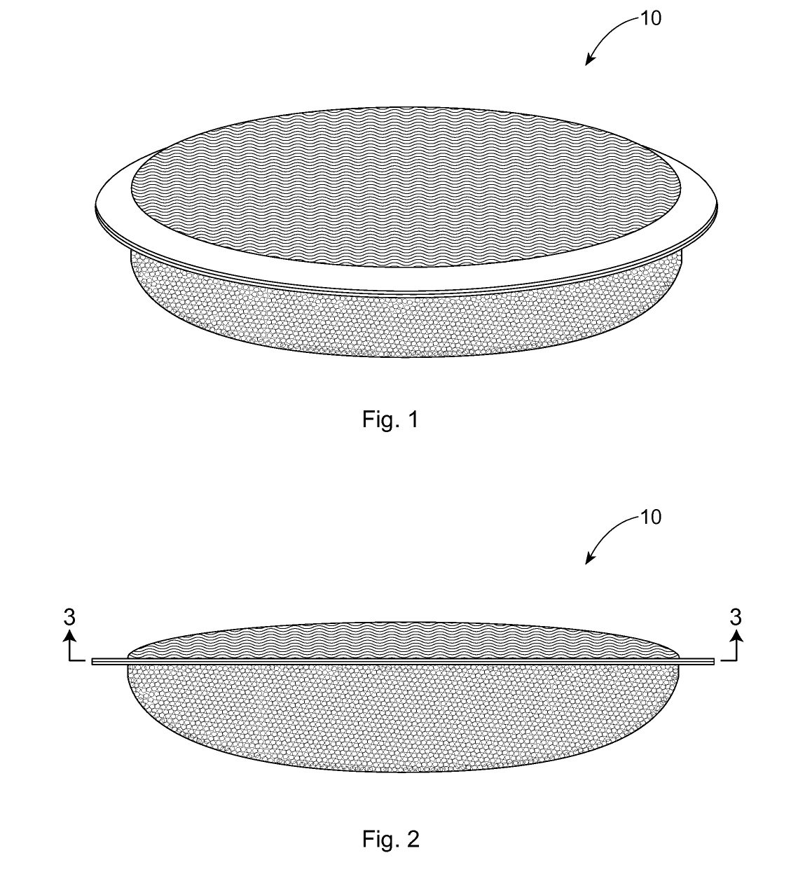

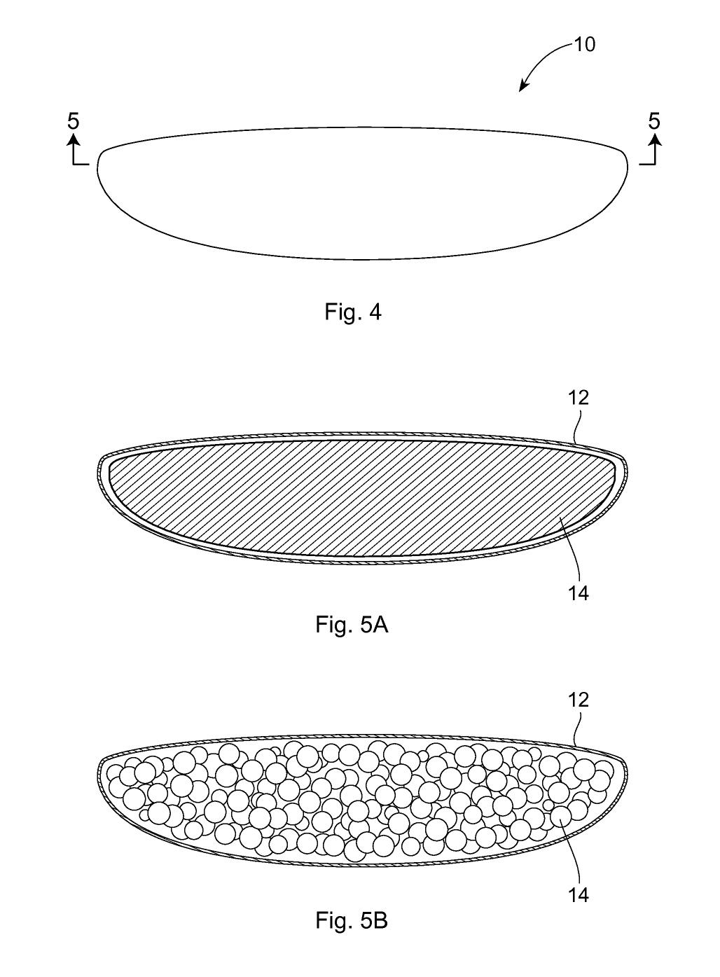

A perspective view of a personal care article 10 according to one embodiment is shown in FIG. 1. As shown in FIGS. 4, 5A, and 5B, the personal care article 10 can comprise a water penetrable first substrate 12 and a personal care composition 14, wherein the water penetrable first substrate 12 is adjacent to the personal care composition 14. The water penetrable first substrate 12 at least partially surrounds the composition 14. In one embodiment, as shown in FIG. 4, a single piece of water penetrable substrate 12 has been wrapped around the personal care composition 14 and sealed (not shown). In FIG. 5B, the composition 14 is in the form of pellets.

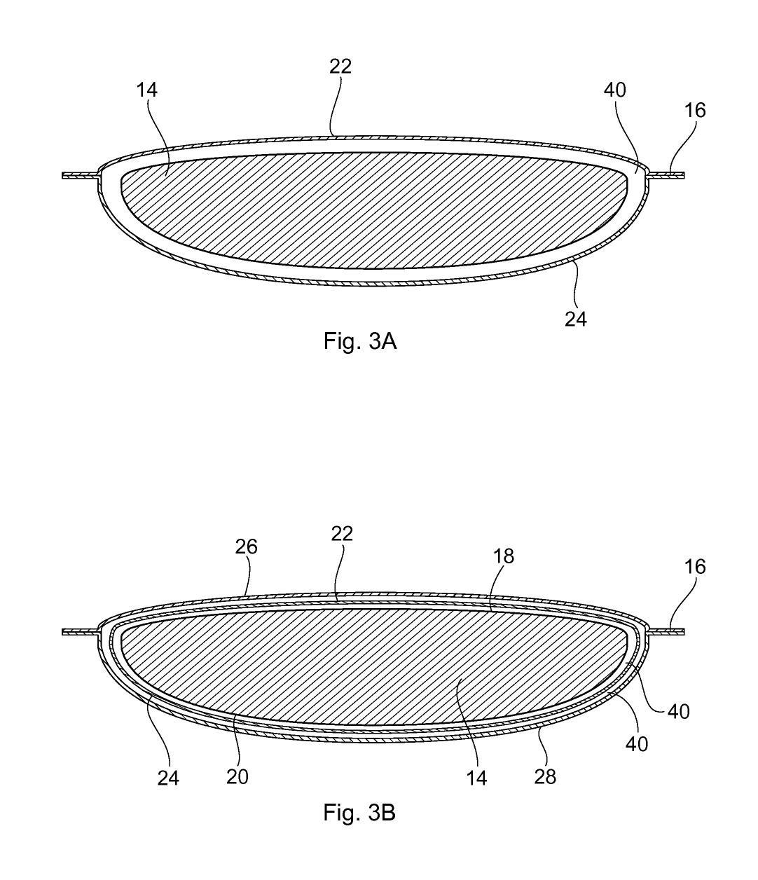

In another embodiment, as illustrated in FIGS. 2 and 3A, a personal care article 10 comprises a personal care composition 14, a first substrate 22 adjacent to the personal care composition 14, and a second substrate 24 adjacent to the personal care composition 14. In one embodiment depicted in FIG. 3A, the seal 16 joining the first and second substrates (22, 24) is only visible on the ends, but actually goes all the way around the personal care composition 14. The first and second substrates (22, 24) may, however, may be sealed in other configurations, or, may only be partially sealed so as to form, for example, a pouch. The first and second substrates (22, 24) may be the same or different.

In another embodiment, as illustrated in FIGS. 2 and 3B, a personal care article 10 comprises a personal care composition 14 having a first side 18 and a second side 20. A first substrate 22 is adjacent to the first side 18, while a second substrate 24 is adjacent to the second side 20. In one embodiment depicted in FIG. 4, the seal 16 joining the first and second substrates (22, 24) is only visible on the ends 30, but actually goes all the way around the personal care composition 14. In addition, a first water insoluble substrate 26 is adjacent to the first substrate 22 and a second water insoluble substrate 28 is adjacent to the second substrate 24. The first and second water insoluble substrates (26, 28) may be the same or different Like the seal of the first and second substrate (22, 24), while only visible on the ends, the seal 16 of the first and second water insoluble substrates (26, 28) goes all the way around the personal care composition 14. The seal 16 of the first and second water insoluble substrate (26, 28) may, however, be sealed in other configurations, or, may only be partially sealed so as to form, for example, a pouch.

The personal care article may also comprise a chamber 40, as seen, for example, in FIGS. 3A and 3B. A chamber is open area between a substrate and a personal care composition or between a substrate and another substrate, where the substrate is not touching the personal care composition or the other substrate. The substrate(s) may be flexible such that they touch the composition (or another substrate) in some areas and not others. The areas where the substrate is touching or not touching the composition or other substrate may shift as the substrate(s) and composition shift during handling and/or use.

The personal care article can include from about 3500% to about 25,000%, by weight of total substrate(s), of a personal care composition. In one embodiment, the article comprises greater than 3,500%, by weight of the total substrate(s), of a composition. In another embodiment, the article comprises greater than 4,000%, by weight of the total substrate(s), of a composition. In varying embodiments, the article comprises greater than 4,250%, by weight of the total substrate(s), of a composition; greater than 4,500%, by weight of the total substrate(s), of a composition; greater than 4,750%, by weight of the total substrate(s), of a composition; greater than 5,000%, by weight of the total substrate(s), of a composition; or any combination thereof.

The personal care article may be in any suitable shape, for example, oval, square, rectangular, circular, triangular, hour glass, hexagonal, c-shaped, etc.

Without wishing to be bound by theory, and as set forth above, it is believed that the personal care article can eliminate problems associated with reusability, waste, consumption, lather, dissolution, and conformity or compliancy. As described herein, it has been found that use of a cleansing composition can improve longevity of a personal care article such that the personal care article can be used for a number of usage events and can conform to or mimic a shape of the surface to which the cleansing composition is applied. Further, it has been found that such a cleansing composition can be combined with a water insoluble substrate made of particular materials to control water flux and, thus, dissolution and consumption of the cleansing composition to increase the longevity of the personal care article without sacrificing lather or other properties desirable to consumers thereof.

Methods for making such personal care articles can include processing a personal care composition. When such personal care compositions have a low enough viscosity, the compositions can be easily pumped and/or transported. To achieve a lower viscosity, such personal care compositions can be, for example, heated. However, a heated personal care composition without necessary rheological properties can flow directly through pores or other openings of a substrate. At a minimum, such heated personal care compositions can otherwise render the personal care article useless by creating a tacky surface. Whereas a small amount of personal care composition can cool and solidify quickly, given the amount of personal care composition used in the personal care article (i.e., about 3,500% or greater, by weight of the substrate, of the personal care composition), thermal inertia can become a concern for a personal care composition without rheological necessary properties as a greater amount of composition can remain heated for a longer period of time.

Without wishing to be bound by theory, it is believed that by providing a personal care composition having a requisite viscosity at a predetermined fill temperature, the personal care composition can be processed as a hot melt and delivered onto a substrate such that the personal care composition can be substantially prevented from flowing through pores or other openings of the substrate. By processing such personal care compositions in the form of a hot melt, the compositions can be more easily pumped and transported, and thus, the personal care article can be made more efficiently and more cost-effectively. This viscosity is known as the complex viscosity which can be measured according to the Oscillatory Rheology Tests as described below. There is a

First Oscillatory Rheology Test for compositions having a viscosity transition temperature up to 95° C. The viscosity transition temperature is the temperature at which the composition is completely flowable. A Second Oscillatory Rheology Test is used for compositions with a viscosity transition temperature above 95° C. The complex viscosity of the composition can be from about 10 Pa·s to about 100,000 Pa·s, from about 10 Pa·s to about 60,000 Pa·s, from about 10 Pa·s to about 30,000 Pa·s, or any combination thereof.

A. Substrates

The personal care article can comprise at least one substrate. The substrate can enhance cleansing and therapeutic treatment of a surface such as skin and/or hair. For example, by physically coming into contact with the skin and/or hair, the substrate can aid in the cleansing and removal of dirt, makeup, dead skin, and other debris such that the substrate can act as an efficient lathering and/or exfoliating implement but can also be non-abrasive to the skin. A substrate can be a composite (i.e. there are multiple plies to the substrate which may be of the same or different materials). In one embodiment, the substrate can be water insoluble. In other embodiments, the substrate can be water penetrable.

In one embodiment, a substrate can at least partially surround a personal care composition. In another embodiment, a substrate can surround a personal care composition. In additional embodiments, a substrate can be in the form of a pouch, pocket, wrap, or a combination thereof.

A substrate can be a contact substrate, which can be a substrate for contacting a target surface, like the skin for example. A substrate can also be a noncontact substrate. Noncontact substrates can, for example, be used to help give a personal care article a desired consumption rate, softness, lather properties, etc.

The substrate can be water penetrable. Where the substrate is water penetrable, the substrate can have a water flux rate. The water flux rate can be used to limit wetting of the cleansing composition included in the personal care article thereby controlling lather, dissolution, and/or consumption of the composition included in the personal care article. Without being limited by theory, the first substrate can manage or limit the water flux rate to provide controlled wetting and to extend a useful life of the personal care composition while still enabling enough wetting to provide, for example, suitable lather. In certain embodiments, the water flux rate can be from about 0.1 cm³/cm²/s to about 200 cm³/cm²/s, from about 0.4 cm³/cm²/s to about 120 cm³/cm²/s, from about 20 cm³/cm²/s to about 100 cm³/cm²/s, or any combination thereof, as measured by the water flux rate test as described in U.S. Patent Application Pub. No. 2012/0246851. The ability to control the water flux rate allows for adjustment such that the composition, like a cleansing composition, can be reused and, thus, last through a number of shower, bathing or cleansing experiences while still exhibiting lathering characteristics expected by consumers.

In some embodiments, there can be a water flux differential between substrates. In varying embodiments, the flux differential between substrates can be at least about 2.5 cm³/cm²/s; about 3.0 cm³/cm²/s or more; or about 4.0 cm³/cm²/s or more.

The substrate can, in some embodiments, need a sufficient tensile strength in order to effectively fulfill its desired role. For example, a contact substrate may need to have a higher tensile strength than a noncontact substrate due to its contact with the target surface. In one embodiment, a substrate can provide an ultimate tensile strength of about 10 g/mm width or greater, about 30 g/mm (width) or greater, about 60 g/mm (width) or greater, or about 200 g/mm (width) or greater and a stiffness of about 1 g/mm (width) or greater, about 2 g/mm (width) or greater, about 7 g/mm (width) or greater, about 20 g/mm (width) or greater, or about 80 g/mm (width) or greater. Tensile strength can be measured according to the Substrate and Article Tensile test in U.S. Patent Application Pub. No. 2012/0246851.

The substrate can further provide a variety of textures. Texturized substrates can be used for both contact and noncontact substrates. In one embodiment, the article can have a different texture on each side thereof. For example, the article can include a gripping side and a substrate application side. In one embodiment, the gripping side can include a texture that is the same as the substrate application side. In another embodiment, the gripping side can include a texture that is different than the substrate application side.

In certain embodiments, the substrate can be a nonwoven (i.e., a natural or synthetic nonwoven including fibrous and nonfibrous nonwovens), a woven, a film (e.g., a formed film), a sponge (e.g., a natural and/or synthetic sponge), a polymeric netted mesh (i.e., a “scrim”), a batting, spunbond, spunlace, hydroentangled, carded, needlepunch, or any other suitable material.

Suitable formed films for use as a substrate in the personal care article can include plastic formed films, such as polyolefins, including, for example, low density polyethylene (LDPE) films, hydroapertured polyethylene films with one or more openings such as apertures of about 3 mm or less, in certain embodiments from about 0.01 mm to about 2 mm, and combinations thereof. Many of such films are available from Tredegar, Inc.

The substrate can also be a nonwoven. A nonwoven can typically have land regions (i.e., regions that do not allow water and/or the cleansing composition to pass through) and openings. In one embodiment, the nonwoven can provide sufficient air space between, for example, openings and land regions of the substrate and can help control permeability of the substrate. The nonwoven substrate can be fibrous or nonfibrous.

Suitable fibrous nonwovens for use as a substrate in a personal care article can include a spunlaid hydroentangled 100% polypropylene (PP) available from Avgol Nonwovens, NC, USA; a carded, calendar bonded all bi-component polypropylene/polyethylene (PP/PE) fiber available from Fiberweb Inc., TN, USA; a spunbond, overbonded 100% PP, and a carded, through air bonded 30/30/40 PP/Bi-component PP-PE/Rayon.

An additional nonwoven suitable for use as a substrate herein can include batting fibers which can include fusible battings. Fusible battings can be fused, for example, by thermoplastic adhesives or via bicomponent fibers. For example, a nonwoven substrate can include a low loft all polyester batting available from Fairfield Processing, Danbury, Conn., USA; a low loft all polyester, ½ thickness (peeled) batting available from Fairfield Processing, Danbury, Conn., USA; a PROEF 12-334 polyester-bicomponent fiber blend batting available from Libeltex, Belgium; a PROEF 12-370 dual layer PET/copet bico and PP fibers available from Libeltex, Belgium; a bulk layer with standard PET/coPET bicotrilobal fibers available from Libeltex, Belgium; a dry web T30 SC batting, hollow PET+bico PET/PE fiber blend, air bonded available from Libeltex, Belgium; a PROEF 12-372 batting, coarse polyester and PE/PET bico fibers available from Libeltex, Belgium; and a dry web T23W batting, coarse polyester and bico fiber mix available from Libeltex, Belgium.

Polymeric netted meshes or scrims can also be useful as a substrate for a personal care article. Some examples can include those described in U.S. Pat. No. 4,636,419. In one embodiment, the substrate can comprise a polypropylene scrim or a polyethylene scrim. In a further embodiment, the substrate can comprise a low density polyethylene scrim.

A substrate can comprise a polymeric mesh sponge. Some suitable polymeric mesh sponges are described in European Patent Application No. EP 702550A1 published Mar. 27, 1996. Polymeric mesh sponges can comprise a plurality of plies of an extruded tubular netting mesh prepared from a strong flexible polymer, such as addition polymers of olefin monomers and polyamides of polycarboxylic acids.

In certain embodiments, a substrate can also be a composite material that includes, for example, one or more plies of the same or different materials such as nonwovens, wovens, films, sponges, scrims, battings, and the like superimposed physically, joined together continuously (e.g., laminated, etc.) in a discontinuous pattern, or by bonding at the external edges (or periphery) of the substrate and/or at discrete loci. The substrate can be a composite material comprising at least one formed film and at least one nonwoven where the substrate can be vacuum-formed. Such a suitable formed film composite material can include, for example, a vacuum-laminated composite formed film material that can be made or formed by combining a carded polypropylene nonwoven having a basis weight of 30 gm² with a formed film.

Additionally, as described above, a substrate can include one or more openings such that water, the composition, and/or lather, for example, can pass through the substrate. In one embodiment, where the permeable substrate can be adjacent to the composition, the water can pass through the water permeable substrate to interact with the personal care composition. As the composition dissolves, it can then also pass through the substrate to be delivered to the target surface, like the skin.

In one embodiment, the permeability of the openings can be selected based on the dissolution half-life of the personal care composition and the desired reusability of the article. For example, when the dissolution half-life of the personal care composition is high, a higher level of permeability can be selected to counteract the high dissolution half-life and provide a desirable consumption rate for the article. Alternatively, when the dissolution half-life of the personal care composition is low, the permeability of the one or more openings can be lower and still provide a desirable consumption rate for the article. In varying embodiments, a substrate can include a permeability of about 1 opening/cm² or greater, about 10 openings/cm² or greater, about 100 openings/cm² or greater, about 500 openings/cm² or greater, about 1,000 openings/cm² or greater, about 1,500 openings/cm² or greater, or any combination thereof.

The openings can be apertured, nonapertured, or a combination thereof. For example, the one or more openings can include well-defined apertures such as microapertures or macroapertures, holes, perforations, cavities, raised or depressed fibrous and/or nonfibrous regions, gaps between regions, and the like that can enable, for example, water and/or the cleansing composition to pass through the substrate.

In one embodiment, a personal care article can comprise more than one substrate. In one embodiment, a personal care article can comprise more than one contact substrate. A combination of contact substrates can be used, for example, to give different properties to different sides of an article. Using FIG. 3B as an example, the first water insoluble substrate 26 can be a contact substrate which helps gripping and the second water insoluble substrate 28 can be a contact substrate on another portion of the article selected for its application properties. As another example, in one embodiment, the article can have an exfoliating contact substrate on one side of the article and a soothing contact substrate on the other side.

A personal care article can also comprise more than one substrate where one substrate comprises a contact substrate and another substrate a noncontact substrate. Using FIG. 3B as an example, the first and second water insoluble substrates 26, 28 would both be contact substrates, while the first and second substrates 22, 24 would be noncontact substrates. In one embodiment, a noncontact substrate can be at least partially surrounded by at least one contact substrate. In another embodiment, two noncontact substrates can be surrounded by two contact substrates. Additional contact and noncontact substrates can also surround other substrates and/or a composition.

A combination of substrates can be used to not only give different user experience properties, like exfoliating versus soothing, but it can also be used to give other desirable properties of an article, like appropriate consumption rate and lather. When combining substrates to form an article, one should consider the properties of the composition, in addition to the individual properties of the substrates, to come up with the article with the desired properties. For example, in one embodiment, a personal care composition can be surrounded by two noncontact substrates which can be surrounded by two contact substrates. In a further embodiment, the two noncontact substrates can be the same. In a further embodiment, the two contact substrates can be the same.

Some examples of suitable substrates are included below, and other suitable substrate materials are also described in Provisional U.S. Patent Application Ser. No. 61/523,824.

B. Personal Care Compositions

As noted above, a personal care article can comprises a substrate and a personal care composition. The personal care composition can be a rinse-off composition (e.g., a cleanser) or a leave-on composition (e.g., a moisturizer).

In one embodiment, the personal care composition can be compliant as discussed above. The personal care composition can have a compliance value of about 0.01-1.50 kg/mm.

In certain embodiments, a personal care composition can have a dissolution half-life. In varying embodiments, the personal care composition can have a dissolution half-life of from about 1.0 min. to about 15 min.; from about 1.1 min. to about 13 min.; from about 1.2 min. to about 12 min.; from about 1.3 min. to about 11 min.; from about 1.4 min. to about 8.0 min.; from about 1.5 min. to about 5 min.; or any combination thereof. The dissolution half-life can be measured according to the Dissolution Rate Test in U.S. Patent Application Pub. No. 2012/0246851.

The personal care composition can be adjacent to one or more substrates. For example, the personal care article can include a composition disposed between the one or more substrates. As shown in FIG. 3A, the cmposition 14 can be disposed within and adjacent to the water penetrable first substrate 12 such that the first substrate 12 can surround the cleansing composition 14. In one embodiment, a substrate can comprise a compartment (e.g., pouch) that can be filled with a cleansing composition. As described herein, the substrate can activate and/or engage the composition.

The personal care composition can be in any suitable form. For example, the personal care composition can be in the form of a bar, paste, gel, pellets, beads, orcombination thereof. Additionally, the composition can be of any shape desirable to a user.

The personal care composition can be, for example, a cleansing composition, a moisturizing composition, a pre-shave composition, a post-shave composition, a shampoo, a conditioner, or a combination thereof. In one embodiment, the personal care composition can comprises a cleansing composition.

In varying embodiments, the cleansing composition comprises a synthetic surfactant, a soap, or a combination thereof. In certain embodiments, the cleansing composition can include one or more of the following: humectants such as glycerin, rheology modifiers such as clays, starches, and polymers, and optional ingredients including emulsifiers such as mono and diglycerides or other low HLB surfactants including nonionic emulsifiers such as polyglycerol esters, benefit agents including antimicrobials such as ZPT or zinc pyrithione, exfoliates, actives, and the like. In one embodiment, a cleansing composition can be a soap-based composition that includes a combination of surfactants(s) and humectant(s) such as glycerin.

A cleansing composition can exhibit a complex viscosity of from about 10 Pa·s to about 100,000 Pa·s; in certain embodiments from about 10 Pa·s to about 60,000 Pa·s; in certain embodiments from about 10 Pa·s to about 30,000 Pa·s; and in certain embodiments from about 10 Pa·s to about 10,000 Pa·s in a temperature range of from about 55° C. to about 125° C. according to the Oscillatory Rheology Tests as described herein. Those with ordinary skill in the art will appreciate that such a temperature range can include a suitable range by which to load and/or combine a substrate with a cleansing composition (this can be referred to as the “fill temperature”). For example, the lower end of the range can signify a cleansing composition with a melting point sufficiently high enough such that the composition will not melt or degrade under standard storage conditions. The high end of the range can signify a temperature for which the substrate does not melt or degrade, thus not affecting the integrity of a personal care article. Thus, in certain embodiments, the fill temperature can be from about 55° C. to about 85° C., and in certain embodiments from about 85° C. to about 125° C. In one embodiment, a cleansing composition can exhibit a complex viscosity of from about 10 Pa·s to about 10,000 Pa·s in a temperature range of from about 85° C. to about 125° C.

In certain embodiments, a cleansing composition can be a complex fluid at a fill temperature. A complex fluid can be translucent or opaque and exhibit non-Newtonian or shear-thinning behavior and can also exhibit a yield stress or a zero shear viscosity that is larger than the viscosity at increased shear rates. Examples of such complex fluids are shown below in Table 4. Additionally, such compositions are likely to form a conformable composition upon cooling. This composition will likely be in the form of a, for example, a conformable mass like a paste where the composition is fairly cohesive to itself and does not penetrate much, if at all, into the substrate.

In certain embodiments, a cleansing composition can include one or more surfactants. Suitable cleansing compositions can include, for example, sodium cocoyl isethionate (SCI) and cocamidopropyl betaine (CAPB). In one embodiment, the cleansing composition can include from about 10% to about 70%, by weight of an active component, of SCI; in certain embodiments, from about 15% to about 65%, by weight of an active component, of SCI; and in certain embodiments from about 20% to about 60%, by weight of an active component, of SCI. Such embodiments can optionally include up to about 10%, by weight of an active component, of CAPB as a cosurfactant; in certain embodiments up to about 8%, by weight of an active component, of CAPB; in certain embodiments up to about 6%, by weight of an active component, of CAPB; in certain embodiments from about 1% to about 7%, by weight of an active component, of CAPB; and in certain embodiments, from about 2% to about 6%, by weight of an active component, of CAPB. In certain embodiments, cosurfactants can improve lathering properties and mildness. Such cleansing compositions can further include glycerin, as described herein, and water. Optional ingredients for such embodiments can include from about 0.5% to about 25% of polyvinyl alcohol, such as CELVOL® 523; from about 0.01% to about 1.0% of polyethylene glycol, such as PEG-23M and PEG-90M; and from about 1% to about 35% of starches. Other optional ingredients can include citric acid and fragrances. Examples of such cleansing compositions are shown below in Table 1.

In one embodiment, the cleansing composition can comprise a synthetic surfactant. In a further embodiment, the surfactant can comprise a mixture of surfactants and the composition can further comprise a rheology modifier.

In one embodiment, a cleansing composition can include from about 1% to about 99.5%, from about 5% to about 85%, or from about 10% to about 70%, by weight of the cleansing composition, of a surfactant or a mixture of surfactants.

Other suitable surfactants for use in the personal care composition can include anionic, nonionic, cationic, zwitterionic, amphoteric surfactants, soap, or combinations thereof. For example, according to one embodiment, the synthetic surfactant can comprise an anionic surfactant. The anionic surfactant can be branched or linear. According to one embodiment, the surfactant can comprise one or more linear anionic surfactants such as ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

In one embodiment, the synthetic surfactant can comprise sodium laureth(n) sulfate, hereinafter SLEnS, where n defines the average moles of ethoxylation. In another embodiment, the synthetic surfactant can comprise sodium trideceth(n) sulfate, hereinafter STnS, wherein n defines the average moles of ethoxylation. According to example embodiments, n for the SLEnS and/or the STnS can range from about 0 to about 8, from about 1 to about 3, about 2, or about 1. It will be understood that a material such as SLEnS or STnS can comprise a significant amount of molecules having no ethoxylate, 1 mole ethoxylate, 2 mole ethoxylate, 3 mole ethoxylate, and so on in a distribution which can be broad, narrow, or truncated. For example, SLE1S can comprise a significant amount of molecules which have no ethoxylate, 1 mole ethoxylate, 2 mole ethoxylate, 3 mole ethoxylate, and so on in a distribution which can be broad, narrow, or truncated and still comprise SLE1S where an average distribution can be about 1. Similarly, ST2S can comprise a significant amount of molecules which have no ethoxylate, 1 mole ethoxylate, 2 mole ethoxylate, 3 mole ethoxylate, and so on in a distribution which can be broad, narrow, or truncated and still comprise ST2S, where an average distribution can be about 2.

The synthetic surfactant can also comprise one or more branched anionic surfactants and monomethyl branched anionic surfactants such as sodium trideceth sulfate, sodium tridecyl sulfate, sodium C₁₂-C₁₃ alkyl sulfate, C₁₂-C₁₃ pareth sulfate, sodium C₁₂-C₁₃ pareth-n sulfate, or combinations thereof.

In other embodiments, a synthetic surfactant can comprise nonionic and cationic surfactants. Nonionic surfactants for use in the cleansing composition can include those selected from the group consisting of alkyl glucosides, alkyl polyglucosides, polyhydroxy fatty acid amides, alkoxylated fatty acid esters, sucrose esters, amine oxides, and mixtures thereof. Cationic surfactants for use in the cleansing composition include, but are not limited to, fatty amines, di-fatty quaternary amines, tri-fatty quaternary amines, imidazolinium quaternary amines, and combinations thereof.

A synthetic surfactant can further include an amphoteric surfactant, a zwitterionic surfactant, or mixtures thereof. According to one embodiment, and like CAPB described herein, the amphoteric and/or zwitterionic surfactants can be combined with other surfactants such that the amphoteric and/or zwitterionic surfactants can be cosurfactants for the cleansing composition. Suitable amphoteric surfactants can include those that are broadly described as derivatives of aliphatic secondary and tertiary amines, in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition can include sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378. In one embodiment, the surfactant included in the personal cleansing composition can comprise an amphoteric surfactant that can be selected from the group consisting of sodium lauroamphoacetate, sodium cocoamphoactetate, disodium lauroamphoacetate disodium cocodiamphoacetate, and mixtures thereof.

Suitable zwitterionic surfactants can include those that are broadly described as derivatives of aliphatic quaternary ammonium, phosphonium, and sulfonium compounds, in which the aliphatic radicals can be straight or branched chain, and wherein one aliphatic substituent contains from about 8 to about 18 carbon atoms and one contains an anionic group (e.g., carboxy, sulfonate, sulfate, phosphate, or phosphonate). In one embodiment, the zwitterionic surfactant included in the personal cleansing composition can comprise one or more betaines such as cocoamidopropyl betaine.

A cleansing composition can further include a rheology modifier. According to one embodiment, the rheology modifier can maintain or provide a particular rheology or viscosity for the cleansing composition.

Suitable rheology modifiers for use in a cleansing composition disclosed herein can include clays, starches, polymers, and the like. Suitable clays for use in the cleansing composition can include both natural and synthetic clays such as montmorillonite and laponite. Additionally, suitable starches for use in the cleansing composition can include carbohydrates such raw starch (corn, sago, tapioca, rice, potato, wheat, and the like), and pregelatinized starch and suitable polymers for use in the cleansing composition can include structuring polymers such as hydrophobically modified polymers, which are described in more detail below.

In another embodiment, a cleansing composition can be a soap-based cleansing composition that can include a combination of a soap or a mixture of soaps suitable for application to a substrate and a humectant such as glycerin or a mixture of humectants.

The cleansing composition of the present invention can typically include from about 40% to about 99.5%, from about 45% to about 75%, or from about 50% to about 65%, by weight of the cleansing composition, of a soap.

A soap can include, for example, alkali metal or alkanol ammonium salts of alkane- or alkene monocarboxylic acids. Sodium, magnesium, potassium, calcium, mono-, di- and tri-ethanol ammonium cations, or combinations thereof, can be suitable. In one embodiment, the soap included in the cleansing composition can include sodium soaps or a combination of sodium soaps with from about 1% to about 25% ammonium, potassium, magnesium, calcium or a mixture of these soaps. In certain embodiments, suitable soaps can include the well-known alkali metal salts of alkanoic or alkenoic acids having from about 12 to 22 carbon atoms, from about 12 to about 18 carbon atoms or alkali metal carboxylates of alkyl or alkene hydrocarbons having from about 12 to about 22 carbon atoms.

A cleansing composition can also include soaps having a fatty acid distribution of coconut oil that can provide a lower end of a broad molecular weight range or a fatty acid distribution of peanut or rapeseed oil, or their hydrogenated derivatives that can provide an upper end of the broad molecular weight range.

In one embodiment, soaps in a cleansing composition can include a fatty acid distribution of tallow and vegetable oil. The tallow can include fatty acid mixtures that typically have an approximate carbon chain length distribution of 2.5% C₁₄, 29% C₁₆, 23% C₁₈, 2% palmitoleic, 41.5% oleic, and 3% linoleic. The tallow can also include other mixtures with similar distribution, such as the fatty acids derived from various animal tallows and lard. In one embodiment, the tallow can also be hardened (i.e., hydrogenated) to convert part or all of the unsaturated fatty acid moieties to saturated fatty acid moieties.

In certain embodiments, the soaps included in a cleansing composition disclosed herein can also include unsaturation in accordance with commercially acceptable standards. For example, in one embodiment, the soaps included in a cleansing composition can include unsaturation in a range of from about 30% to about 50% of saponified material; and in certain embodiments, from about 37% to about 45% of saponified material.

The cleansing composition can further include a humectant such as glycerin. In one embodiment, the humectant can maintain or provide a particular rheology or viscosity for the cleansing composition such that the cleansing composition can be manipulated to conform to or mimic the surface of the substrate to which the personal care article is being applied.

In one embodiment, a cleansing composition can include from about 5% to about 90%; in certain embodiments from about 5% to about 80%; in certain embodiments from about 10% to about 65%, by weight of the cleansing composition, of a humectant, such as glycerin or a mixture of humectants. In one embodiment, the cleansing composition can include from about 10% to about 65%, by weight of an active ingredient, of glycerin. Other suitable humectants can include sorbitol, propylene glycol, butylene glycol, hexylene glycol, ethoxylated glucose, 1,2-hexane diol, hexanetriol, dipropylene glycol, erythritol, starch, trehalose, diglycerin, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, salts such as chlorides, sulfates, carbonates, and combinations thereof.

A cleansing composition disclosed herein can also include one or more optional ingredients such as polymers, gums, Pluronics®, inorganic salts such as zinc carbonate, antimicrobial agents, actives, brighteners, silica, moisturizers or benefit agents, and emulsifiers as described below.

In one embodiment, the cleansing composition can include one or more polymers. The one or more polymers can structure the cleansing composition, modify the rheology of the cleansing composition, and improve lather, skin feel, and/or deposition of benefit agents or antimicrobial agents included in the cleansing composition disclosed herein, and the like. In one embodiment, the one or more polymers can be hydrophobically modified polymers or other suitable structuring polymers, cationic polymers, deposition polymers, and the like. For example, in one embodiment, the cleansing composition can optionally include from about 0.01% to about 10%, from about 0.1% to about 8%, or from about 0.1% to about 5%, by weight of the cleansing composition, of a hydrophobically modified polymer.

For example, in one embodiment, the cleansing composition can optionally include from about 0.01% to about 10%, from about 0.1% to about 8%, or from about 0.1% to about 5%, by weight of the cleansing composition, of a hydrophobically modified polymer. Suitable hydrophobically modified polymers can include Acrylates/Vinyl Isodecanoate Crosspolymer (Stabylen 30 from 3V), Acrylates/C10-30 Alkyl Acrylate Crosspolymer (Pemulen TR1 and TR2), Ammonium Acryloyldimethyltaurate/Beheneth-25 Methacrylate Crosspolymer (Aristoflex HMB from Clariant), Acrylates/Beheneth-25 Methacrylate Copolymer (Aculyn 28 from Rohm and Haas); Acrylates/Steareth-20 Methacrylate Copolymer (Aculyn 22 from Rohm and Haas), PEG-150/Decyl Alcohol/SMDI Copolymer (Aculyn 44 from Rohm and Haas), PEG-150 Distearate (Aculyn 60 from Rohm and Haas), Acrylates/Steareth-20 Methacrylate Crosspolymer (Aculyn 88 from Rohm and Haas), Aqupec SER-150 (acrylates/C10-30 alkyl acrylates crosspolymer) comprising about C18 (stearyl) side chains and about 0.4% hydrophobic modification (HM), Aqupec HV-701EDR which comprises C8 (octyl) side chains and about 3.5% HM., Stabylen 30 (from 3V Sigma S.p.A), and Carbopol Aqua SF-1 (crosslinked acrylates copolymer) having average 4.5 carbon alkyl side chains and more than 50% HM, or combinations thereof.

A cleansing composition can also optionally include cationic polymers to improve the lathering and skin feel benefits of the cleansing composition during and after use. If present, a cleansing composition can include from about 0.001% to about 10%, from about 0.01% to about 5%, or from about 0.05% to about 1%, by weight of the composition, of a cationic polymer. Suitable cationic polymers for use in the cleansing composition disclosed herein can include, but are not limited to, cationic polysaccharides; cationic copolymers of saccharides and synthetic cationic monomers; cationic polyalkylene imines; cationic ethoxy polyalkylene imines; cationic poly[N-[3-(dimethylammonio)propyl]-N′[3-(ethyleneoxyethylene dimethyl ammonio)propyl]urea dichloride]. Suitable cationic polymers can include polymers having a quaternary ammonium or substituted ammonium ion.

In certain embodiments, a cleansing composition can optionally include from about 0.001% to about 10%, from about 0.01% to about 5%, or from about 0.05% to about 1%, by weight of the cleansing composition, of a deposition polymer. Suitable deposition polymers can include cationic hydroxyethyl cellulosic polymers (polyquaternium 10, UCARE polymer JR400, LR400, JR30M, KG30M), cationic guar polymers (JAGUAR® from Rhodia (Jaguar C135, Jaguar C145, Jaguar C-17, Hi-Care 1000, Jaguar Excel, Jaguar CHT), and N-HANCE® polymers from Aqualon (N-Hance 3000, N-Hance 3196, N-Hance 3198, N-Hance 3205, N-Hance 3215, N-Hance 3269, N-Hance 3270), and synthetic acrylamide polymer (polyquaternium 76), and combinations thereof.

In one embodiment, the cleansing composition can include from about 5% to about 50%, from about 10% to about 40%, or from about 20% to about 30%, by weight of the cleansing composition, of a block copolymer. Examples of a block copolymer can include, for example, an F87, F88, F98, F108, or F127 Pluronic®, or a combination thereof.

A cleansing composition can optionally further include one or more antibacterial agents that can serve to further enhance antimicrobial effectiveness of the cleansing compositions. If present, the cleansing composition can include from about 0.001% to about 5%, from about 0.01% to about 2%, from about 0.1% to about 1%, or from about 0.2% to about 0.5%, by weight of the cleansing composition, of the one or more antimicrobial agents. Other examples of antibacterial agents can include the carbanilides, for example, triclocarban (also known as trichlorocarbanilide), triclosan, a halogenated diphenylether available as DP-300 from Ciba-Geigy, hexachlorophene, 3,4,5-tribromosalicylanilide, and salts of 2-pyridinethiol-1-oxide, salicylic acid and other organic acids, and a pyrithione or a polyvalent metal salt of pyrithione such as a zinc pyrithione. Other suitable antibacterial agents are described in detail in U.S. Pat. No. 6,488,943.

The cleansing composition can optionally further include a benefit agent. Suitable benefit agents can include, for example, petrolatum, mineral oil, soy bean oil, paraffin, polyethylene, polybutene, polydecene, dimethicone, alkyl siloxanes, cyclomethicones, lanolin, sucrose polyesters, or combinations thereof.

Moisturizers can also optionally be included in a cleansing composition to provide skin conditioning benefits and to improve mildness of the personal care article. A selection of levels and types of moisturizers to be incorporated into the cleansing composition can be made without adversely affecting a stability of the cleansing composition or its in-use characteristics, thereby delivering good moisturization and lather.

Examples of suitable moisturizers can include long chain fatty acids, liquid water-soluble polyols, glycerin, propylene glycol, sorbitol, polyethylene glycol, ethoxylated/propoxylated ethers of methyl glucose (e.g., methyl gluceth-20) and lanolin alcohol (e.g., Solulan-75). Such moisturizers can be used at a concentration or level of from about 2% to about 20%, from about 4% to about 15%, or from about 8% to about 12%, by weight of a cleansing composition.

A cleansing composition can also include one or more emulsifiers. For example, a cleansing composition can include from about 0.01% to about 10%, from about 0.1% to about 8%, from about 0.5% to about 5%, or from about 1% to about 3%, by weight of the cleansing composition, of an emulsifier. Suitable emulsifiers can include mono and diglycerides or other low HLB surfactants including nonionic emulsifiers such as polyglycerol esters.

### III. PROCEDURES

A. Oscillatory Rheology Tests
1. First Oscillatory Rheology Test

To measure the viscous (G″) and elastic (G′) moduli (loss and storage modulus) or factors and complex viscosity (n*) of a personal care composition with transition temperatures lower than 95° C., use a AR G2 Rheometer (TA Instruments, DE, USA) equipped with cross hatched lower and upper geometry, where a diameter of an upper geometry is 40 mm. Measurement can be conducted by placing approximately 5 grams of personal care composition onto a lower test geometry and manually applying force to create an even flat surface. The sample can be compressed until the upper geometry is in full contact with personal care paste and a gap can be about 1,000 microns. Conduct the oscillatory test over a temperature range of about 25° C. to about 90° C. at a frequency of 1.0 Hz using a constant oscillatory stress of 75 Pa utilizing a temperature ramp of 5° C. per minute.

The temperature range may be adjusted for compositions which would degrade if run up to 90° C. by stopping at a point between the transition temperature and the degradation temperature.
2. Second Oscillatory Rheology Test

To measure the viscous (G″) and elastic (G′) moduli (loss and storage modulus) or factors and complex viscosity (n*) of a personal care composition at temperatures greater than 95° C., use an AR 2000 Rheometer (TA Instruments, DE, USA) equipped with parallel plate lower geometry and cone upper geometry, where a diameter of an upper geometry is 25 mm and the cone angle is 5 degrees. The rheometer should be equipped with a TA environmental test chamber with a suitable supply of liquid nitrogen. Measurements can be conducted by placing approximately 5 grams of personal care composition onto a lower test geometry and manually applying force to create an even flat surface. The sample can be compressed until the upper geometry is in full contact with cleansing paste and a gap can be about 150 microns. Conduct the oscillatory test over a temperature range of about 40° C. to about 120° C. at a frequency of 1.0 Hz using a constant oscillatory stress of 75 Pa utilizing a temperature ramp of 10° C. per minute. The temperature range may be adjusted for compositions which would degrade if run up to 120° C. by stopping at a point between the transition temperature and the degradation temperature.

B. Viscosity Test

To measure the viscosity of personal care compositions of a personal care article at temperatures greater than 95° C., use an AR 2000 Rheometer (TA Instruments, DE, USA) equipped with parallel plate lower geometry and cone upper geometry, where a diameter of an upper geometry is 25 mm and the cone angle is 5 degrees, the gap between upper and lower geometry can be about 150 microns. If below 95° C., use an AR G2 Rheometer (TA Instruments, DE, USA) equipped with cross hatched lower and upper geometry, where a diameter of an upper geometry is 40 mm, the gap between upper and lower geometry can be about 1000 micron. Condition the sample for about 2:00 min at the desired measurement temperature prior to starting the steady state flow test. Conduct the steady state flow test from 1.0 to 500 l/sec taking 10 data points per decade.

C. Compliance Test

To measure the compliance of a personal care composition and/or article, use a Texture Analyzer TA-XT2i (Texture Technologies Corp, NY, USA) equipped with at least a 5 kg load cell and a 0.75 inch ball probe at ambient conditions, with the probe zero point at an article top surface using 0.5 gram-force to register a probe height, and a 2 gram-force to commence data collection for both force and distance. Measure a compressive force (kg) at a compression rate of 1 mm/sec over a depth of 5 mm, ensuring that the personal care composition and/or article form a flat surface over contact area with the ball probe, near the center of the article or composition. Repeat measurements as needed (e.g. at least 3 times) to obtain a representative average value. To determine the compliance of the composition and/or article divide the maximum observed force (kg) by the maximum compression depth (5 mm). When using a 5 kg load cell some samples may exceed capacity, in this case the maximum compression depth will be less than the set depth of 5 mm, specified in the procedure.

### IV. METHODS OF MAKING PERSONAL CARE COMPOSITIONS AND PERSONAL CARE ARTICLES

In certain embodiments, personal care articles can be manufactured by combining a personal care composition as described herein with a sheet or pouch (formed from a substrate described herein) via a conventional method which can include, but is not limited to, spraying, slot coating, molding such as rotary molding, extrusion, feeding from a hopper and cutting such as by wire cutting, and roll transfer (e.g., pressure roll).

In certain embodiments, a personal care composition can be prepared by combining the raw components of a personal care composition to form a slurry and then processing the slurry through a mill to obtain a paste-like substance. In another embodiment, the slurry can be processed in an extruder, which can provide intimate mixing at lower temperatures. In another embodiment, powder and liquid components can be combined and heated (e.g., at 180° C. or greater) such that the components are melted into a paste-like substance.

To further prepare the personal care composition for delivery onto the substrate, the paste-like substance can be heated to a hot melt paste such that the personal care composition can be pumped and processed more easily. In one embodiment, a heat exchanger can be used to regulate the temperature of the hot melt. The fill temperature, as described herein, can be from about 55° C. to about 125° C. In certain embodiments, the fill temperature can be from about 60° C. to about 110° C. In other embodiments, the paste-like substance or slurry can be pumped and processed at higher viscosities through the use of an extruder, scraped wall heat exchanger, or other suitable processing techniques.

In certain embodiments, one or more pouches or compartments can be formed within a first sheet of a substrate, by a thermoforming process, a vacuum-forming process, or a combination thereof. Such processes are described herein, and greater detail can be found in U.S. Pat. No. 7,386,971 and European Patent No. 01161370.

In one embodiment, when using an indexing thermoforming process, a first sheet of a substrate can be drawn over a forming die having plurality of forming cavities. In one embodiment, each cavity can possess a dome shape with a round edge, such that the edges can be substantially free of any sharp edges that can damage the film during forming or sealing. Further, each cavity can include a raised surrounding flange. A sheet of the substrate can be heated by, for example, a heating plate that can be positioned to superpose the forming die. Such heating plate can include a plurality of concave depressions which can correspond to the cavities on the forming die. In one embodiment, a vacuum can be pulled through the pre-heating plate to ensure intimate contact between the sheet of the substrate and the heating plate to provide uniform heating. Non-uniform heating can result in a substrate of a personal care article having weak spots.

The thermoformed sheet can be molded into the cavities forming a plurality of compartments. In one embodiment, compartments, once formed, can retain their thermoformed orientation by applying a vacuum through walls of the cavities. Once the compartments can be formed, a personal care composition can be added to each compartment. A second sheet can then be superposed on the first sheet to cover the filled compartment and then the first sheet and the second sheet can be heat sealed using a heating plate. In one embodiment, raised flanges surrounding each cavity can ensure that the sheets are sealed together along the flange to form a continuous closed seal. Once sealed, the personal care articles can be separated using cutting means, as described below.

In a continuous vacuum-forming process, for example, a first sheet can be continuously fed onto a sealing surface having one or more molds, wherein the sealing surface can be moved along with the first sheet. In one embodiment, the sealing surface can have a horizontal motion for a period of time such that the one or more compartments can be formed on the first sheet, the one or more compartments can be filled with the personal care composition, a second sheet can be superposed on and sealed to the first sheet, and resulting personal care articles can be cut and separated. As such, the surface can be a part of and/or removably connected to a moving, rotating belt, such as a conveyor belt or a platen conveyor belt.

A sealing surface can be connected to one or more vacuum systems. In one embodiment, a first vacuum system can provide an under-pressure continuously on the area between or along the molds/edges and a second vacuum system can only provide a vacuum for a certain amount of time, to draw the first film into the molds. The molds can comprise a variety of shapes, lengths, widths, and depths, depending on the required dimensions or configurations of a personal care article.

In certain embodiments, a first sheet can be fed to a sealing surface continuously at a constant speed throughout the process. This can be done by any known method such as, for example, using rollers from which the first sheet can unwind. In such embodiments, the first sheet can be guided by a guide device (e.g., a belt). Further, once on the sealing surface, the first sheet can be held in position by any known means such as grips or clips on the edges of the sealing surface, where there are no molds, or pressed down with rollers on the edges of the sealing surface, where there are no molds, or held down by a belt on the edges of the surface, where there are no molds.

For ease of operating and film positioning and for improved accuracy and better alignment reliability, a first sheet can be held in position by application of a vacuum, thus drawing or pulling the first sheet in a fixed position on the sealing surface. This can be done by applying a vacuum (or under-pressure) through the sealing surface to hold the first sheet. The vacuum can be applied along the edges of the first sheet, and thus, typically the edges of the surface and/or on the surface area between or around the molds and along the edges thereof.

In certain embodiments, compartments can be formed using a vacuum or under-pressure to draw a first sheet into the molds. In one embodiment, each mold can comprise one or more holes which can be connected to a system which can provide a vacuum through these holes and onto the first sheet above the holes. In certain embodiments, heat can be applied to the first sheet and/or the first sheet can be wetted.

In one embodiment, compartments can be filled by flood dosing, wherein the first sheet can pass with continuous horizontal or substantially horizontal motion under a dosing unit which can be static and can comprise a device to accurately dose a set amount or volume of product per time unit. In another embodiment, the compartments can be filled using continuous motion in-line filling, which can use a dispensing unit, positioned above an open end of the compartments, having an endless, rotating surface with nozzles. In one embodiment, the rotating surface can rotate with continuous motion, at a same speed as the compartments, such that the open end of each compartment can be under the same nozzle or nozzles for the duration of the filling step. A nozzle or set of nozzles can be connected to a device which can accurately control an amount or volume of personal care composition that can be dispensed during a rotation (e.g., in one compartment). For example, a filling unit can use a flow meter and/or positive displacement pump to dose correct amounts or volumes of personal care composition. Such correct amounts or volumes of personal care composition can be pumped and fed to the nozzles. Other methods can include using flow measurement, by use of a magnetic flow meter or mass flow meter, and pressure flow filling/measurement.

Filled compartments can then be sealed. In one embodiment, the filled compartments can be sealed while in a horizontal position and in continuous, constant motion. In such an embodiment, a second sheet can be continuously fed over and onto the first sheet having open, filled compartments and then the first sheet and the second sheet can be sealed together. The first sheet and the second sheet can be sealed in an area between the molds, and thus, between compartments. In one embodiment, the second sheet can be fed onto the first sheet with a same speed and moving in a same direction as the first sheet.

Sealing can be performed continuously and with a constant speed by methods including heat sealing, solvent welding, and solvent or wet sealing. In embodiments where heat sealing can be applied, a roller having a pattern and size of the personal care articles can be rolled over the sheets, such that the heated roller can contact only a sealing area, namely between the compartments and around edges of the molds. In other embodiments, a movable, returnable sealing device can contact areas between the molds, around the edges, for a certain time, to form the seal and then move/rotate away from the sealing area. In embodiments where wet sealing or welding is used, heat can be applied. For example, solvent can be selectively applied to an area between the molds or on the second film by, for example, spraying or printing, and then pressure and/or heat can be applied to such areas, to form the seal.

The sealed compartments on the film can then be cut by a cutting device (e.g., die cutter), and the cutting can be performed using a variety of known methods. In certain embodiments, and as with the processes described above, cutting can also be performed in a continuous manner. The cutting device can be a sharp item or a hot item. In one embodiment, the cutting device can comprise a roller with sharp tools, such as knives, with cavities of the size and pattern of the personal care articles, such that the roller can only contact those areas which are intended to be cut.

The sheets can be sealed together by a conventional sealing method which may include, but is not limited to, heat, pressure, glue, ultrasound, etc. Sealed sheets can then be partitioned into units for a consumer's use. Optional manufacturing steps may include calendaring to flatten the article as well as drying, cooling, printing, and punching to produce orifices.

### V. METHODS OF USE

A method of cleansing the skin and/or hair with a personal care article can include wetting with water a reusable personal care article and contacting the skin or hair with the wetted personal care article.

The personal care articles can be intended to be wetted with water prior to use. The personal care article can be wetted by immersion in water or by placing the personal care article under a stream of water. In one embodiment, lather can be generated from the personal care article by mechanically agitating and/or deforming the personal care article either prior to or during contact of the personal care article with the skin and/or hair. The resulting lather can be useful for cleansing the skin and/or hair. During a cleansing process and subsequent rinsing with water, any therapeutic or aesthetic benefit agents can be deposited onto the skin and/or hair. Deposition of the therapeutic or aesthetic benefit agents can be enhanced by physical contact of the substrate with the skin and/or hair as well by the inclusion of one or more deposition aids.

### VI. EXAMPLES

A. Personal Care Composition Examples A-T

Compositional information for Examples A-T can be found in Table 1. The compositions for Examples A-T were prepared by combining each of respective ingredients listed for Examples A-T, and then mixing them in a planetary Kitchen Aid mixer and stiffing until a homogeneous slurry was formed. The homogeneous composition was then transferred to an industrial 3-roll mill, and milled thrice until the composition is paste-like (slurry) with no lumps or particles. The resultant paste-like composition was then heated to a predetermined temperature (e.g., 85° C.) to form a hot melt. The complex viscosity was then measured and results are provided in Table 2.

TABLE 1

| | | | | | | | | |
|---|---|---|---|---|---|---|---|---|
| Ex- | | Glyc- | Wa- | | PEG | Citric | | Fra- |
| ample | SCI | erin | ter | CAPB | 90 M | Acid | Starch | grances |

Amts. by wt. % of active component

| | | | | | | | | |
|---|---|---|---|---|---|---|---|---|
| Ex. A | 39.15 | 35.10 | 14.94 | 3.60 | 0.10 | 0.00 | 0.00 | 1.25 |
| Ex. B | 41.50 | 31.30 | 14.00 | 6.00 | 0.08 | 0.00 | 0.00 | 0.94 |
| Ex. C | 41.50 | 30.80 | 14.00 | 6.00 | 0.10 | 0.15 | 0.00 | 1.25 |
| Ex. D | 39.15 | 33.50 | 14.00 | 6.00 | 0.10 | 0.15 | 0.00 | 1.25 |
| Ex. E | 37.41 | 35.50 | 14.00 | 6.00 | 0.10 | 0.15 | 0.00 | 1.25 |
| Ex. F | 26.19 | 24.85 | 9.80 | 4.20 | 0.07 | 0.11 | 30.00 | 0.88 |
| Ex. G | 36.21 | 38.49 | 12.95 | 5.55 | 0.09 | 0.14 | 0.00 | 1.16 |
| Ex. H | 31.32 | 46.80 | 11.20 | 4.80 | 0.08 | 0.12 | 0.00 | 1.00 |
| Ex. I | 32.59 | 34.64 | 11.66 | 5.00 | 0.08 | 0.12 | 10.00 | 1.04 |
| Ex. J | 28.19 | 42.12 | 10.08 | 4.32 | 0.07 | 0.11 | 10.00 | 0.90 |
| Ex. K | 24.66 | 48.11 | 8.82 | 3.78 | 0.06 | 0.09 | 10.00 | 0.79 |
| Ex. L | 35.59 | 40.66 | 12.01 | 5.15 | 0.09 | 0.13 | 0.00 | 1.07 |
| Ex. M | 32.03 | 36.59 | 10.81 | 4.63 | 0.08 | 0.12 | 10.00 | 0.96 |
| Ex. N | 30.25 | 34.56 | 10.20 | 4.37 | 0.07 | 0.11 | 15.00 | 0.91 |
| Ex. O | 28.47 | 32.53 | 9.60 | 4.12 | 0.07 | 0.10 | 20.00 | 0.86 |
| Ex. P | 26.69 | 30.49 | 9.00 | 3.86 | 0.06 | 0.10 | 25.00 | 0.80 |
| Ex. Q | 24.91 | 28.46 | 8.40 | 3.60 | 0.06 | 0.09 | 30.00 | 0.75 |
| Ex. R | 27.41 | 53.45 | 9.80 | 4.20 | 0.07 | 0.11 | 0.00 | 0.88 |
| Ex. S | 23.49 | 60.10 | 8.40 | 3.60 | 0.06 | 0.09 | 0.00 | 0.75 |

B. Viscosity Measurements

Complex viscosities for each of Examples A-T can be found in Table 2. Complex viscosity measurements were taken for each of the above examples at 55° C. and 85° C. As set forth herein, measurements taken at temperatures below 95° C. are taken using the First Oscillatory Rheology Test described above.

TABLE 2

Complex Viscosity (Pa · s)

| Example | 55° C. | 85° C. |
|---|---|---|
| Ex. A | 23,880 | 17 |
| Ex. B | 64,860 | 2,794 |
| Ex. C | 106,000 | 8,279 |
| Ex. D | 89,480 | 7,768 |
| Ex. E | 6,900 | 15 |
| Ex. F | 39,330 | 7,908 |
| Ex. G | 28,630 | 1,147 |
| Ex. H | 8,013 | 16 |
| Ex. I | 23,860 | 556 |
| Ex. J | 9,552 | 49 |
| Ex. K | 1,603 | 10 |
| Ex. L | 32,640 | 1,307 |
| Ex. M | 33,940 | 1,323 |
| Ex. N | 23,810 | 918 |
| Ex. O | 28,490 | 1,692 |
| Ex. P | 24,780 | 1,667 |
| Ex. Q | 16,890 | 1,960 |

Complex viscosities for each of Examples G-I and R-T can be found in Table 3. Complex viscosity measurements were taken for each of the above examples at 105° C. and 115° C. As set forth herein, measurements taken at temperatures above 95° C. are taken using the Second Oscillatory Rheology Test described above.

TABLE 3

Complex Viscosity (Pa · s)

| Example | 105° C. | 115° C. |
|---|---|---|
| Ex. G | 0.53 | — |
| Ex. H | 0.23 | 0.18 |
| Ex. I | 0.60 | 0.42 |
| Ex. R | 0.17 | 0.16 |
| Ex. S | 0.16 | 0.12 |
| Ex. T | 0.26 | 0.22 |

Table 4 shows viscosity measurements for each of Examples D-E and G-H at shear rates of 1.0 s⁻¹ and 100 s⁻¹. As set forth above, complex fluids can be translucent or opaque and exhibit non-Newtonian or shear-thinning behavior and can also exhibit a yield stress or a zero shear viscosity that is larger than the viscosity at increased shear rates. Examples of such complex fluids are shown below in Table 4.

TABLE 4

| Viscosity (Pa · s) | |
|---|---|
| 85° C. | 105° C. |

| Example | at 1.0 s⁻¹ | at 100 s⁻¹ | at 1.0 s⁻¹ | at 100 s⁻¹ |
|---|---|---|---|---|
| Ex. D | 116 | 0.83 | — | — |
| Ex. E | 43.9 | 0.72 | — | — |
| Ex. G | — | — | 3.78 | 0.45 |
| Ex. H | — | — | 12.5 | 1.02 |

C. Examples of Substrates
1. Formed Films

| | | Caliper and | Pore count/ | Water Flux | Air |
|---|---|---|---|---|---|
| | | Basis | area; and | Rate | Permeability |
| Code | Material Description | Weight | diameter | cc/cm²/s | m³/m²/s |
| F1 | Hydroapertured | 166 | 1,780/cm² | 6.2 | 58 |
| | polyethylene film on 100 | microns, | — | | |
| | mesh screen, white | 24.5 gsm | | | |
| | (Tredegar Inc.) | | | | |
| F2 | Vacuum formed | 560 | 115/cm² | 33.8 | 295 |
| | polyethylene film, white | microns, | — | | |
| | (SSRIS-CPM, Tredegar | 24.5 gsm | | | |
| | Inc.) | | | | |
| F3 | Vacuum formed | 560 | 91/cm² | — | 130 |
| | polyethylene film, white 22 | microns, | ~500 | | |
| | Hex (Tredegar, Inc.) | 24.4 gsm | micron | | |
| F4 | Vacuum formed | 935 | 22.2/cm² | — | 145 |
| | polyethylene film, blue 11.2 | microns, | 1.1 mm | | |
| | Hex (Tredegar, Inc.) | 29.4 gsm | | | |
| F5 | Vacuum formed | 670 | 49/cm² | — | 220 |
| | polyethylene film, green | microns, | 0.9 mm | | |
| | (Tredegar, Inc.) | 36.0 gsm | | | |
| F6 | Vacuum formed | 33.5 gsm | 12.6/cm² | — | — |
| | polyethylene film, white | — | 1 mm | | |
| | (Tredegar, Inc.) | | | | |
| F7 | Vacuum formed | 418 | 285/cm² | 11.5 | 16.2 |
| | polyethylene film 40 Hex | microns, | — | | |
| | | 35.8 gsm | | | |

Caliper: ASTM D645
Air Permeability: ASTM D737

2. Fibrous Nonwovens

| | | | Water Flux |
|---|---|---|---|
| | | Basis | Rate |
| Code | Material Description | Weight | cc/cm²/s |
| N1 | Spunlaid hydroentangled 100% PP (Avgol | 47 gsm | 6.0 |
| | Nonwovens, NC, USA) | | |
| N2 | Carded, calendar bonded all bicomponent | 32 gsm | 20.7 |
| | PP/PE fiber | | |
| | (Fiberweb Inc., TN, USA) | | |
| N3 | Spunbond, overbonded 100% PP | 37 gsm | 2.1 |
| | (Experimental nonwoven) | | |
| N4 | Carded, through air bonded 30/30/40 | 62 gsm | 2.8 |
| | PP/Bicomponent PP-PE/Rayon (calendar | | |
| | patterned) | | |

3. Fibrous Nonwoven Battings

| | | Caliper; | Water |
|---|---|---|---|
| | | and Basis | Permeability |
| Code | Material Description | Weight | cc/cm²/s |
| B1 | Quilter's Fusible batting, low loft all | 2.50 mm, | 58.3 |
| | polyester (Fairfield Processing, | 160 gsm | |
| | Danbury, CT, USA) | | |
| B2 | Quilter's Fusible batting, low loft all | 1.21 mm, | 71.3 |
| | polyester, ½ thickness (peeled) | 80 gsm | |
| B3 | PROEF 12-334 polyester-bicomponent | 1.54 mm, | — |
| | fiber blend batting (Libeltex, | 100 gsm | |
| | Belgium) | | |
| B4 | PROEF 12-370 dual layer PET/copet | 0.60 mm, | — |
| | bico and PP fibers; bulk layer with | 55 gsm | |
| | standard PET/coPET bico trilobal | | |
| | fibers (Libeltex, Belgium) | | |
| B5 | Dry Web T30 SC batting, hollow PET + | 0.41 mm, | — |
| | bico PET/PE fiber blend, through air | 35 gsm | |
| | bonded (Libeltex, Belgium) | | |
| B6 | PROEF 12-372 batting, coarse | 0.55 mm, | — |
| | polyester and PE/PET bico fibers | 50 gsm | |
| | (Libeltex, Belgium) | | |
| B7 | Dry Web T23W batting, coarse | 0.56 mm, | — |
| | polyester and bico fiber mix (Libeltex, | 50 gsm | |
| | Belgium) | | |

Caliper measured at 0.8 grams/mm²

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care article, the personal care article comprising:
a) a substrate; and
b) about 3,500% to about 10,000%, by weight of the substrate, of a personal care composition, the personal care composition surrounded by the substrate, wherein the personal care composition exhibits a complex viscosity from about 10 Pascal-seconds (Pa·s) to about 100,000 Pa·s and has a compliance value of about 0.01 kg/mm to about 1.5 kg/mm.

2. The personal care article of claim 1, wherein the composition is in the form of a paste.

3. The personal care article of claim 1, wherein the personal care composition exhibits a complex viscosity from about 10 Pa·s to about 60,000 Pa·s.

4. The personal care article of claim 1, wherein the personal care composition exhibits a complex viscosity from about 10 Pa·s to about 30,000 Pa·s.

5. The personal care article of claim 1, wherein the personal care composition is a complex fluid in the temperature range from about 55° C. to about 125° C.

6. The personal care article of claim 1, wherein the substrate is formed from at least one of a nonwoven, a woven, a film, a sponge, a polymeric netted mesh, and a batting.

7. The personal care article of claim 1, wherein the substrate comprises apertures such that an average aperture size is about 3 mm or less.

8. The personal care article of claim 7, wherein the average aperture size is from about 0.01 mm to about 2 mm.

9. The personal care article of claim 1, wherein the article comprises about 4,000% or greater, by weight of the substrate, of the personal care composition.

10. The personal care article of claim 1, wherein the article exhibits a compliance value of about 0.01 kg/mm to about 1.50 kg/mm.

11. A method of making a personal care article, the method comprising:
a) providing a substrate, wherein the substrate comprises a compartment having an open end;
b) filling the compartment with about 3,500% or greater, by weight of the substrate, of a personal care composition via the open end; and
c) sealing the open end such that the personal care composition is surrounded by the substrate;
wherein the personal care composition exhibits a complex viscosity from about 10 Pascal-seconds (Pa·s) to about 100,000 Pa·s and has a compliance value of about 0.01 kg/mm to about 1.5 kg/mm.

12. The method of claim 11, wherein the compartment comprises a pouch, such that the personal care composition is loaded into the pouch and then sealed.

13. The method of claim 11, wherein a second substrate is placed over the initial substrate when the open end is sealed to form the personal care article.

14. The method of claim 11, wherein the method uses an indexing process to form the personal care article.

15. The method of claim 11, wherein the method uses a continuous process to form the personal care article.

16. The method of claim 11, wherein the substrate is formed from at least one of a nonwoven, a woven, a film, a sponge, a polymeric netted mesh, and a batting.

17. The method of claim 11, wherein the substrate comprises apertures such that an average aperture size is about 3 mm or less.

18. The method of claim 11, wherein the average aperture size is from about 0.01 mm to about 2 mm.

19. The method of claim 11, wherein the composition is in the form of a paste.
